(19) United States
(12) Patent Application Publication
(10) Pub. No.: US 2003/0059483 A1
(43) Pub. Date: Mar. 27, 2003

(54) Color stable hypochlorous sanitizer and methods

(76) Inventors: Eddie D. Sowle, Woodbury, MN (US); Darryl C. Bowling, Greensboro, NC (US)

Correspondence Address: MERCHANT & GOULD PC, P.O. BOX 2903, MINNEAPOLIS, MN, 55402-0903, US

(21) Appl. No.: 09/501,876

(22) Filed: Feb. 10, 2000

Related U.S. Application Data
(63) Continuation-in-part of application No. 09/257,086, filed on Feb. 24, 1999, now abandoned.

(51) Int. Cl. A61K 33/14

(52) U.S. Cl. 424/661; 424/662; 424/663; 252/186.35; 424/405

(57) ABSTRACT

Manual warewashing in common food service locations is typically performed in a multicompartment sink within three or more basins. In one basin the ware is contacted with an aqueous solution of a detergent composition. The ware is cleaned using mechanical action to remove soil. The cleaned ware is often rinsed in a subsequent sink in a potable water rinse and is then sanitized in a sanitizing solution in a subsequent basin, typically the third sink in sequence. The sanitizing solution can be rapidly depleted during periods of large volumes of hand washed ware. In order to monitor and control the concentration of the sanitizer in the sanitizer sink, we have found that even highly oxidizing hypochlorite bleaches, if adjusted to an appropriate pH, can maintain substantial sanitizing capacity while not decolorizing otherwise oxidatively sensitive dyes. In the method of the invention, the sanitizing solution adjusted to a pH of less than about 7 containing a dye can maintain a stable dye solution for a period of time greater than the time required to deplete 90% or more of the OCl⁻¹ in solution. As a result, the hand washing staff can have a satisfactory indication of the existence of active sanitizer in the sanitizer step based on the presence of color in the aqueous solution.

### RELATED APPLICATIONS

This application is a continuation-in-part application of U.S. Ser. No. 09/257,086 filed Feb. 24, 1999.

### FIELD OF THE INVENTION

The invention relates to a solid concentrate composition which is shelf stable for a minimum of two years. The invention also relates to a liquid or solid composition that combine a dye and a chlorine source resulting in unique cleaning or sanitizing properties with controlled, measured, acceptable and useful chlorine stability. The invention also relates to methods for cleaning or sanitizing hard surfaces and for hand washing ware in a multibasin sink using at least a washing step involving an aqueous detergent solution followed by a sanitizing step involving an aqueous chlorine based sanitizer solution. The invention further relates to a spray bottle application for sanitizing hard surfaces.

### BACKGROUND OF THE INVENTION

Active halogen, e.g. chlorine, materials have been available for bleaching, sanitizing and cleaning purposes for many years. Such materials in the form of hypochlorite (NaOCl), chlorinated isocyanurate compounds, encapsulated chlorine sources, chlorinated tripolyphosphate, etc. have been used in single solutions or more commonly in alkaline, aqueous, powdered or solid materials to form active concentration of chlorine. Such materials are commonly used to bleach clothing, clean or sanitize hard surfaces, and other generic destaining, antimicrobial or soil removing processes.

Cleaning solutions using surfactants, builders, detergents, etc. for removing soil or the reduction of microbial populations on hard surfaces have been in use for many years. Such hard surfaces include ceramic, metal, plastic composite, surfaces that can be walls, floors, countertops, tables, chairs, food surface apparatus, etc. Such surfaces come into contact with a variety of soils and can also promote the growth of large populations of microorganisms. The removal of such soils and the reduction of microbial populations is an important goal in maintaining a high quality food service operation.

Another important type of hard surface is the surface of ware including dishware tableware and kitchenware. The hand washing of dish and kitchenware is commonly achieved in a multibasin sink by first contacting soiled ware in an aqueous detergent solution with hand or mechanical agitation for the purpose of removing soil from the ware. Such processes can also include other steps such as a prescraping step, a deliming step, a stain bleaching step or other conventional operations. Once cleaned of soil, the ware is thoroughly rinsed typically with potable water. Once rinsed, the ware is then submerged in a sanitizing bath in a third basin and allowed to drain and permitted to dry. Such a sanitizing step ensures that microbial populations are substantially reduced.

One common use for chlorine based sanitizer solutions is in a final sanitizing step in a hand surface sanitizing or warewashing method using a solution made by diluting commonly available aqueous sodium hypochlorite. Dilution ratio of about 1 part by volume of sodium hypochlorite per 10,000 parts of service water are used resulting in an effective bleaching and sanitizing solution at a strength of greater than 100 parts per million, or for certain applications, 50 parts per million (ppm) active chlorine. The conventional solutions typical in the prior art have a substantial hypochlorite (OCl⁻¹) concentration and an alkaline pH. Such a sanitizing solution is highly effective in bleaching stains and is very effective at reducing microbial populations. Such solutions can also be used on hard surfaces for soil, stain and microbial control.

These sanitizing solutions are used until depleted of an effective chlorine content and are replaced when the concentration of the oxidizing species drops below a certain concentration typically below about 50 ppm active chlorine. Maintaining an effective concentration of the oxidizing species in the final sanitizing solution is important to maintain cleanliness, sanitization and a stain free condition in the ware. Active chlorine or OCl⁻¹ concentration is typically monitored using indicator strips or test kits. Oxidizing solutions are highly active and can oxidize and decolorize a dye, used at conventional concentration, contained in the solution, rapidly often in an amount of time less than about 15 minutes. Since dyes are typically used at very low concentrations, the substantial decolorization of the solution consumes little hypochlorite but provides little information with respect to the concentration of the hypochlorite in solution. Dishwashing or kitchen personnel cannot know when to change the chlorine depleted solution to maintain at least 50 ppm active chlorine. As a result, the sanitizing solution is discarded and replenished very often resulting in substantial waste of materials, time and money. Potentially worse is the situation in which the solution is not changed often enough, resulting in inadequate sanitization due to an active chlorine concentrate less than 50 ppm.

Attempts have been made to produce stabilized colored or dye containing hypochlorite materials. Initial efforts using inorganic insoluble pigments were attempted. Other attempts are shown, for example, in Jones et al., U.S. Pat. No. 4,554,091, which discloses a colored polymer latex material. The latex tends to form an organic phase separate from the aqueous phase resulting in reduced decolorization in a hypochlorite bleaching composition. Rapisarda et al., U.S. Pat. No. 5,089,162, teach a bleach stable dispersible soluble yellow colorant. Rapisarda et al. disclose that a granular liquid or gelled warewashing detergent comprising a source of alkalinity such as a silicate, a builder, a surfactant and other warewashing components can be made stable in the presence of 0.01 to about 5% of available chlorine from a chlorine bleach and a specifically disclosed yellow colorant or dye. Choy et al., U.S. Pat. No. 5,376,297 disclose thickened aqueous hard surface cleaning compositions containing a colloidal alumina thickener in combination with hard surface cleaner compositions such as a surfactant, a buffer, solvents, etc. The thixotropic hard surface cleaner contains a source of oxidative chlorine and can contain a dispersible pigment. Wise, U.S. Pat. No. 5,384,061, discloses an aqueous thickened liquid or gel typically automatic warewashing detergent composition and can contain a dye in the presence of sodium hypochlorite. However, Choy and Wise fail to disclose the sanitizing of ware in a third sink basin.

Kitko, U.S. Pat. No. 4,248,827, discloses a toilet sanitizing composition which produces hypochlorite ion in solution and contains a water soluble bleachable dye that provides a transitory visual signal. The dye is oxidized to a colorless state within 5 seconds to 15 minutes. Cosentino et al., U.S. Pat. No. 5,279,735, discloses a stable colored peracetic acid solution which contains a dye indicating its presence. Sumi et al., JP 91-200365, disclose a detergent composition that cleans and sanitizes in a single step and develops color upon dilution. Color duration is controlled by dye concentration, which results in solution color lasting from 2 to 12 minutes depending on solution temperature.

A substantial need exists for sanitizing materials that contain active halogen sources and a stable dye. In use, the stable dye may act as an indicator of active halogen concentration or content. The formulation, dye type and constituent concentrations can be adjusted such that the presence of color is indicative of a proper sanitizing solution. As the bleaching, sanitizing, cleaning properties of the sanitizer use solution is consumed over a useful period of time, the solution loses color indicating the possible consumption of active chlorine and the need for a new sanitizer use solution. A further need for acid powdered materials having a chlorine source in a stable dye that can be diluted into a use solution having the unique chlorine indicator is a long term goal of the industry. Further, a substantial need exists for improving methods using chlorine-containing sanitizing solutions in such a way that the solution can contain a stable soluble dye material that is sufficiently stable, (i.e.), a detectable color for a period after a substantial portion, (e.g.) of the chlorine based species have been depleted from the sanitizing solution but an effective amount of chlorine remains when the solution is replaced. Restaurant personnel need to know when a reasonable time has elapsed, indicating that a new solution is needed in order to maintain proper sanitization. Such a time period to be useful is no less than 15 to 30 minutes and is typically greater than 2 but less than 24 hours, preferably greater than 2 but less than 6 hours.

### BRIEF DISCUSSION OF THE INVENTION

We have found a unique liquid, solid unit or powdered composition comprising an encapsulated source of halogen, preferably chlorine, and an indicator dye formulated such that a use solution made by diluting the liquid or powdered composition results in an aqueous composition containing an active concentration of a halogen source that can be gauged, estimated or monitored by the depth of color in the solution. We have also found a unique liquid, solid unit or powdered composition comprising a source of acid, an encapsulated source of halogen, preferably chlorine, and an indicator dye formulated such that a use solution made by diluting the liquid or powdered composition results in an acidic aqueous composition containing an active concentration of a halogen source that can be monitored by the depth of color in the solution. In the solid unit, powdered or solid concentrate form, effervescing tablet and/or solid block, this composition is shelf or storage stable for a minimum of two years. We have also found that the depth of color in such aqueous solutions can be used as an indicator of concentration of the active halogen species. Lastly, we have found a number of methods using such solid unit. powdered and liquid materials.

More specifically, we have found a hard surface cleaning or a hand warewashing method or process including a sanitizing step in which a chlorine based sanitizing solution with a dye used in the sanitizing step. The sanitizer can be formulated with an active chlorine source and sufficient dye to survive a predetermined period. The sanitizing solution made from the composition of the invention can also be stabilized using a near neutral or an acid pH in such a way that a soluble dye added to the sanitizer solution can survive and provide color to the sanitizing solution for a predetermined period of time. Such a period of time is an amount of time sufficient to deplete greater than 50%, 60%, 75%, 90% or other predetermined depletion target for the chlorine based oxidant in the sanitizer solution. This means that after a period of time when the solution goes clear or changes color, (i.e.), no original color present, a replenishment of the active chlorine or a new sanitizer use solution is required. The loss or change in color indicates that the concentration of halogen has been reduced significantly and can be reduced to near ineffective levels. This indicates the need for a fresh solution. Monitoring the solution color will allow the personnel to know at all times that a proper sanitizing solution is present. In the sink sanitizer basin, two to six hours is adequate and is a reasonable predetermined period of time. In the other uses including hard surface cleaning three to twenty four hours is adequate and is a reasonable predetermined period of time. The length of time between the formation of the solution and the depletion of color can be adjusted by adjusting dye concentration and other active ingredient concentrations in the solid unit, powder or liquid material. The aqueous chlorine containing solutions of the invention can be prepared in two specific embodiments. In a first embodiment, the active chlorine solution can be prepared with any arbitrary pH. Often such pH's are mildly or strongly alkaline. In such a case, an amount of dye is used such that the color of the solution is maintained, even in the presence of the active chlorine sanitizer for a predetermined period. The rates of reaction between the dye and the chlorine based sanitizer can be easily measured at a defined alkaline pH and an amount of dye is added to the composition to ensure that the dye survives to the end of the predetermined period. Once the dye in the solution is depleted, then the solution can be replaced or refreshed with additional chlorine source and dye. We have also found in an alternate mode, that if used at a near neutral or an acid pH (pH less than about 7) that the dyes are unusually stable. In such a mode, a substantially reduced dye concentration can be used while maintaining an effective color in the sanitizing solutions for the predetermined period of time. We have also found, at active pH, that the active chlorine species possess enhanced antimicrobial activity or sanitizing capacity. Whereas, in alkaline chlorine species, an effective killing of microorganisms can exist at concentrations of between 100 and 1000 ppm, at acid pH's the concentration of the material can be reduced as low as 50 ppm with maintaining effective antimicrobial action.

Such materials can be used in a variety of useful processes that use the unique qualities of the halogen source. Generally such processes involve removing stains, removing soil, or killing microbial populations on surfaces that require cleaning. In a multiple sink method of warewashing, ware is commonly washed in a first sink with aqueous detergent and exposed to mechanical action to remove soil resulting in cleaned ware. After the first sink the ware can optionally be treated in subsequent sinks for a variety of purposes. Then the cleaned ware is rinsed in a potable water rinse and is contacted with the dye containing chlorine sanitizer in a subsequent sink or basin for sanitizing purposes.

In a hard surface method, the hard surface is contacted with the oxidative halogen bleach composition in an overall cleaning method. The hard surface can be scraped, washed with a detergent solution, rinsed and sanitized with the solutions of the invention. In this method, the solutions are diluted and placed into an applicator bottle having the dye visible through a translucent or transparent bottle. The material is applied preferably with a spray device uniformly contacting the hard surface with 50 to 200 ppm of the active halogen sanitizer material. The sanitizer can be wiped from the surface or simply allowed to dry.

The preferred oxidative halogen chlorine-based sanitizing solution comprises a major proportion of an aqueous medium, a soluble oxidative active chlorine or chlorine based sanitizer, and a soluble organic dye. In one embodiment said solution is maintained at a pH less than about 7, preferably between a pH of 2 to 6.5. One solution that maximizes chlorine activity and user comfort obtains about 90 to 200 ppm active Cl₂ at a pH of about 5.5 to 7. At such a preferred pH, the concentration of hypochlorous acid (HOCl) is maximized while the concentration of hypochlorite (OCl⁻¹, usually NaOCl) is minimized. Such a solution can be made from a powdered or solid concentrate or liquid co-systems comprising a diluent, a dye, a chlorine source and other ingredients including an acid or acid salt. We have found that hypochlorite not hypochlorous acid is the major oxidative species that decolorizes dye in hypochlorite based sanitizers. As a result, the change in pH permits the dye to survive a substantial period since the oxidative (OCl⁻¹) species is at reduced concentrations when compared to alkaline (pH>8) solutions. While the strength or capacity for the solution to remove surface stains in the ware is somewhat reduced, the ability to sanitize ware surfaces is substantially increased. As a result of this pH modification of the sanitizing solution, the dye can survive an extended period of time in the sanitizing solution. The dye can be selected and matched with an appropriate pH such that the dye is depleted of color after a reasonable amount of time, roughly simultaneously with the sanitizing solution being substantially depleted of oxidizing chlorine species. However, preferably the sanitizing solution remains at least some detectable color until the oxidative chlorine species is depleted or consumed by bleaching or sanitizing processes.

For the purpose of this patent application, the term “ware” indicates dishware, pots and pans, flatware, glassware, metallic and plastic utensils, and other tools and containers common in institutional or commercial kitchen or restaurant environments. For the purpose of this patent application, the term “solid unit” refers to a circular, cylindrical, pyramidal, rectangular, octangular or other geometrically shaped solid block or object having a mass of at least 1 gram, preferably 5-25 grams. The term “solid unit” does not refer to a particulate or granulated solid or simple high viscosity liquids that retain some shape. The term “subsequent basin” means that the basin follows the previous basin. However, one or more basins can come between the first basin and a subsequent basin to provide other method steps prior to the sanitizing step. Typically, the sanitizing basin is the last basin in the process. After ware contact with the sanitizing solution, the ware is typically not further contacted with an aqueous solution because even service water can contain some level of a microbial population that can contaminate the sanitized surface.

One aspect of the invention is a method of using a color stable hypochlorous acid sanitizer material in a mode that permits the operator to gauge the bleaching or sanitizing capacity of a hypochlorous acid sink contents using a dyestuff. In this aspect the quantity of dye combined with the active chlorine material in the claimed compositions is matched to the pH and chlorine concentration to give apparent or detectable dye color to the hypochlorite solution for a predetermined time. After the dye color disappears or is depleted, the active chlorine can be replaced or augmented with an added active chlorine and dye composition.

A second aspect of the invention is a chemical composition that can be used to form the color stable hypochlorous acid sanitizer materials used in the method discussed above. Such compositions comprise an active chlorine source and a dye in an amount that can give apparent or detectable dye color to the hypochlorite solution for a predetermined time, such time selected to ensure at least 50 ppm active chlorine is present in the solution. After the dye color disappears or is depleted, the active chlorine can be replaced or augmented with added chlorine composition.

A third aspect of the invention is a is a solid unit in the form, for example, of a tablet or pellet composition that can be manufactured and used to form the aqueous color stable hypochlorous acid sanitizer materials of the compositions and in the methods set forth above. Simple solid units such as tablets or pellets can be formulated to contain the active ingredients of the stable system. In use, to create an active chlorine aqueous system or to replenish an aqueous system during operations, one or more pellets or tablets of the active materials can be introduced into the appropriate sink or container to create the active materials. Surprisingly, we have found that certain forms of preferred dyes are compatible in long term storage in the presence of highly active chlorine based oxidizing agents or sanitizers. After the dye color disappears or is depleted, the active chlorine can be replaced or augmented with added chlorine composition.

### DETAILED DISCUSSION OF THE INVENTION

The invention resides in a solid unit, liquid or powdered and solid compositions comprising a source of halogen and a dye. The composition can contain an acid source to maintain the pH<7. The invention also resides a method for hand washing or cleaning ware in a step-wise fashion with a sanitizing step as a last step in the method. Typically, the first step in such a method involves contacting ware with an aqueous solution of a detergent composition for the purpose of removing soil from the surface of the ware. The invention also resides in a method for cleaning hard surfaces. The cleaning step reduces microbial population substantially in a sanitizing fashion. Typically the first step in such a method involves rinsing or scraping the hard surface followed by an application of the sanitizing material. The sanitizing material can be left in place to dry or can be rinsed or wiped from the surface.

The sanitizing solution can contain an effective concentration of one or more active and inactive ingredients that interacts with the ware and soil to enhance the ability of the aqueous medium to remove soil species. The ware can be exposed to mechanical action by dishwashing personnel who use pads, brushes, scrapers, etc. to remove soil. The aqueous detergent solution can be maintained at a high temperature (40-80° C.) to promote the cleaning action of the aqueous detergent. Such solutions are often replaced periodically when the detergent action is depleted by the presence of substantial quantities of proteinaceous and oily or fatty soils. Prior to contacting the ware in such an aqueous detergent step, the ware is often scraped, rinsed or pretreated to promote soil removal in the detergent step. Following the initial cleaning step, the ware can be rinsed in a potable water rinse to remove the remaining aqueous detergent solution that can contain some small proportion of soil.

After the rinse step, the ware can be contacted with a variety of different compositions in subsequent sinks or basins. One common step is a deliming step for the purpose of removing hard inorganic calcium or magnesium based coatings from the ware comprising hardness, cations and other materials in a film or coating. Such a step is often an acid deliming step that can substantially brighten and clarify the appearance of glassware. The ware can also be contacted in an aqueous rinse composition in a rinse station. Such rinse compositions contain organic polymeric agents that promote rinsing of the ware. A variety of other stations or steps can be used in the method for the purpose of providing enhanced cleaning, brightening the appearance of the glass or metal ware, preserving the color or appearance of plates and cups, destaining tea stains or coffee stains from coffee mugs or cups or a variety of other operational steps.

Halogen Source or Chlorine Sanitizer

The hard surface or the ware is contacted with a sanitizing solution commonly comprising an active halogen or chlorine, based sanitizer composition. The sanitizing solution is typically made from a solid unit, solid, powdered or liquid concentrate of a chlorine containing product by dissolving the material in water. One preferred solid chlorine concentrate of the invention contains a powdered or granular dye, a particulate encapsulated chlorine source, an acid or acid salt dispersed in a substantially neutral alkali metal salt acting as a diluent or extender. Useful salts include sodium sulfate, sodium phosphate, sodium chloride, and other similar available extender salt materials. Sources of halogen, chlorine, used in the methods of the invention include oxidizing compositions capable of liberating an active halogen species, typically Cl₂ or OCl⁻¹ or equivalent materials. Suitable agents for use in the present methods include both liquids and solid forms of halogen preferably chlorine sources, for example, chlorine containing compounds such as solutions of chlorine, hypochlorite, chloramine, etc. Preferred halogen releasing compounds include the alkali metal hypochlorite, alkali metal dichloroisocyanurate, chlorinated trisodium phosphate, monochloramine and dichloramine and the like. Encapsulated chlorine sources may also be used having at least one encapsulating layer surrounding a core of a chlorine source. Such encapsulated chlorine sources have multiple encapsulating layers. Encapsulated chlorine source are disclosed in U.S. Pat. No. 4,618,914 and 5,213,705.

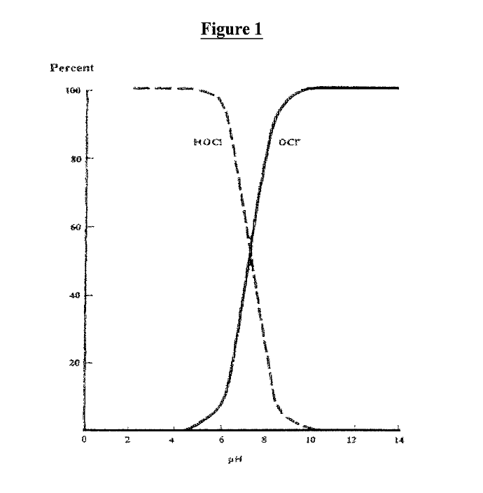

The most common chlorine based sanitizer composition comprises sodium hypochlorite derived form an encapsulated source or from aqueous hypochlorite or other liquid and powdered or solid chlorine sources. Aqueous hypochlorite is typically sold in the form of an aqueous solution containing approximately 5-10 wt-% sodium hypochlorate. Solid sources of chlorine include chlorinated isocyanurate powder or encapsulate. Such materials, having a high pH, can be diluted with water to form an oxidizing aqueous solution containing an oxidative species at a concentration of about 50 to about 300 ppm, preferably about 60 to 200 ppm most preferably 70 to 150 ppm of the oxidative species. Depending on pH, there is an equilibrium (see FIG. 1) between hypochlorous acid and hypochlorite according to the following general equilibration reaction in formula I:

HOCl⇄OCl⁻¹+H⁺¹. (I)

In a common ion effect, as the acid concentration of the solution is increased, the equilibrium of this reaction is pushed towards producing a substantial proportion of hypochlorous acid while minimizing the concentration of hypochlorite. The pH driven concentration relationship between hypochlorous acid (HOCl) and hypochlorite (OCl⁻¹) is shown is FIG. 1. An optimum pH, for conservation of dye, is found where the concentration of the hypochlorous acid is maximized while the concentration of hypochlorite is minimized. Preferably, the concentration of (HOCl) is greater than about 80 percent, while the concentration of (OCl⁻¹) is less than about 20 percent.

The method of the invention uses an aqueous sanitizing composition containing an oxidative chlorine bleach. The aqueous rinse used in the method can be manufactured by diluting a liquid co-system, powdered, pelletized or solid chlorine bleach containing composition. Preferably, the composition contains a chlorine source, the soluble dye, optionally an acid source that is typically diluted by a liquid or solid diluent or stabilizer. In practicing the process of the invention, sufficient amount of a liquid or powdered concentrate is added to the sanitizing process basin. The material dissolves in the aqueous liquid, creating an effective concentration of HOCl and dye at an appropriate pH. The aqueous solution is used until the color is depleted and is replaced when needed.

The oxidative chlorine concentrate of the invention can contain either a liquid or solid source of halogen, liquid sources of halogen, bleach commonly comprise alkali metal such as sodium hypochlorite bleach. These materials are commonly available in aqueous solution in a variety of concentrations. A variety of solid chlorine sources are also available such as chlorinated sodium tripolyphosphate, solid dichloroisocyanurate, calcium hypochlorite and others. Such oxidizing agents are disclosed in Kirk-Othmer, Encyclopedia of Chemical Technology, Second Edition, Volume III, pp. 550-566. A preferred source of chlorine comprises an encapsulated chlorine source. Such chlorine sources are shown in Olson et al., U.S. Pat. Nos. 4,681,914 and 5,358,635.

The chlorine releasing substances suitable as the core material of the encapsulated active chlorine compound include chlorine components capable of liberating active chlorine species such as a free elemental chlorine or OCl⁻, under conditions normally used in warewashing processes. Useful inorganic sources of chlorine include solid materials that yield hypochlorite in aqueous environments including lithium hypochlorite, calcium hypochlorite, etc. Useful organic chlorine releasing compounds must be sufficiently soluble in water to have a hydrolysis constant (K) of about 10⁻⁴ or greater. Those with K values below 10⁻⁴ do not produce sufficiently high concentration of free available chlorine or other active chlorine species for effective bleaching. In general, hydrolysis constants of the N-chloro compounds range from 10⁻¹⁰ to approximately 10⁻³. The principle N-chloro compounds used in bleaching are the chlorinated isocyanurates, which are chlorimides.

Sodium dichloroisocyanurate dihydrate, a preferred chlorine releasing substance suitable as the core substance of the present encapsulated active chlorine compound, is commercially available from Olin Chemicals, Stamford, Conn., as CDB-56™; or as ACL-56™; Monsanto Company, St. Louis, Mo. The chemical structure of this compound is represented by the formula (II) below:

MCl₂(NCO)₃.2H₂O (II)

wherein M is an alkali metal such as Na+, K+, etc.

The encapsulate typically has one, two or more coatings sufficient to reduce chlorine loss. The innermost, chlorine releasing core of the encapsulated active chlorine compound of the present rinse aid concentrate is surrounded by an intermediate coating or spacer layer. This intermediate coating is preferably inorganic and can comprise a filler or builder compound (or mixtures thereof) and provides a protective barrier or spacing between the innermost chlorine core and the organic or inorganic outer layer(s). The outer layer can comprise inorganic builders or organic surfactants.

The encapsulated halogen source is present in the concentrate at a concentration of about 1 to 90 wt-%, preferably about 5 to 70 wt-%.

Acid Source

The chlorine concentrate compositions of the invention are typically combined with an acid source to provide in the final sanitizing solution a pH of less than about 7 to control and minimize the concentration of OCl⁻¹ and maximize the concentration of HOCl. Generally, any normally liquid or normally solid acid source which will facilitate the formation of such low pH may be used in the composition of the invention. A liquid aqueous material can contain either solid or liquid acid. Both organic and inorganic acids have been found to be generally useful in the present composition. Organic acids useful in accordance with the invention include hydroxyacetic (glycolic) acid, citric acid, formic acid acetic acid, propionic acid, butyric acid, valeric acid, caproic acid, gluconic acid, and itaconic acid, trichloroacetic acid, benzoic acid, among others. Organic dicarboxylic acids such as oxalic acid, malonic acid succinic acid, glutaric acid, maleic acid, fumaric acid, adipic acid, terephthalic acid among others are also useful in accordance with the invention. Any combination of these organic acids may also be used intermixed or with other organic acids which allow adequate formation of the composition of the invention. Inorganic acids useful in accordance with the invention include phosphoric acid, sulfuric acid, sulfuric acid, methylsulfamic acid, hydrochloric acid, hydrobromic acid, and nitric acid among others. Powdered acid salts can also comprise a source of acid for the invention. Such acid salts can comprise sodium hydrogen sulfate, sodium dihydrogen phosphate, monosodium citrate, monosodium tartrate, monosodium succinate and other similar powdered acid salt compositions. These acids may also be used in combination with other inorganic acids or with those organic acids mentioned above. Preferred acids for a powdered composition are solid or powdered inorganic or organic acid. The acid source is present in the concentrate at a concentration of about 0 to 30 wt-%, preferably about 0.5 to 30 wt-%, most preferably 5 to 15 wt-%. The chlorine concentrate of the invention can also contain common builders in an acid form such as sodium sulfate (Na₂SO₄), sodium carbonate (NaCO₃), trisodium phosphate, sodium bicarbonate (NaHCO₃) and other acid builder salts such as sodium dihydrogen phosphate, disodium hydrogen phosphate, potassium hydrogen tartrate, monosodium nitrilo triacetic acid and other such acid salts that can aid in forming an appropriate acid pH, provide mild buffering action and aid in sanitization. The acid builder salts are present in the concentrate at a concentration of about 0 to 90 wt-%, preferably about 5 to 75 wt-%.

Dye or Indicator

The sanitizing solution and the chlorine containing concentrate of the invention include a dye. Such dyes can comprise common ordinary dyes or can also include indicator dye materials. Dyes are typically intensely colored substances used at low concentration with a coloration of various substances. The visual properties of dyes are determined by their electronic transitions within the dye molecule. The shade or hue of the dye is determined by energy differences between states in the molecular orbitals. A large number of dyes of varying properties are known. Dyes useful in this invention are typically acid compatible dyes that are stable in the presence of HOCl at the pH disclosed in the invention. Dyes that may have utility in the invention include anthraquinone dyes. Useful dyes include such species as blue tetrazolium dye, brilliant blue G, brilliant blue R, brilliant cresol blue, brilliant sulfone red, brilliant yellow, bromcresol green, reactive blue No. 2, reactive red No. 2, reactive yellow No. 2, FD&C No. 40, FD&C No. 3, etc. Preferably the dye is selected for ease in blending with the powdered chlorine source, the acid salts and the diluent or extender of the invention. However, the dye should be used at a concentration such that the dye begins to fade as the concentration of the OCl⁻¹ begins to be depleted from the sanitizing solution, while the HOCl concentration remains. We have found that the particle size of the dye material is important to maintain dye stability in the tablet or solid concentrates of the invention. We have found that the dye particle having a particle size greater than 200 microns, preferably greater than about 400 microns, most preferably greater than 600 microns, can be made in the form of a solid, powder or a solid unit concentrate and is stabilized to contact with the encapsulated chlorine source. Such a result is surprising in light of the highly active oxidizing capacity of the chlorine source and the sensitive nature of typical organic dye molecules.

The sanitizing solution can contain an organic indicator dye. Such substances reveal through color changes the degree of acidity or basicity of a solution. Most indicators are weak organic acids or bases which exist in one or more structural form (tautomers) of which at least one form is colored. In the case the indicator dye has two colored species, the colors are substantially different and can be detected in solution. Intense colors are desirable so that the minimum concentration of indicator can be used. Depending on the nature of the equilibration reaction between colored species and the uncolored species or between species of different color, the color can occur at a characteristic pH for each indicator. Care must be taken to use an indicator having an appropriate pH change. Indicator dyes that can be used in the context of this invention include methyl violet, metacresol purple, thimole blue, tropeoline 00 (orange roman IV), bromphenol blue, methyl orange, bromcresol green, methyl red, orthophenol red, bromcresol purple and others that have substantial color within the pH of about 3 to about 7. Typically, the sanitizing solution is free of any component that can react with the oxidizing species. However, the sanitizing solution can contain other materials that can enhance the antimicrobial properties or the bleaching properties of the sanitizing solution. Such materials include other oxidative species, oxidation promoters, etc.

The dye is present in the concentrate at a concentration of about 0.001 to 0.5 wt-%, preferably about 0.05 to 0.3 wt-%. Depending on the type of system used, the amount of dye is selected to ensure that the dye provides detectable color for the predetermined period, which period typically ensures that the solution contain at least 50 ppm active chlorine or, depending on circumstances, greater than about 100 ppm active chlorine. One of ordinary skill in the art will have no trouble in formulating these materials with the appropriate amount of dye since the rate of reaction of dye with the chlorine species selected can be easily determined for the purpose of selecting dye concentrations for the concentrate materials. We have found that the amount of dye needed for acid based sanitizer materials is roughly 10% of the amount required to maintain color in neutral or alkaline systems.

Aqueous Detergent

The ware is contacted, in the method of the invention, in a first basin or sink containing an aqueous detergent composition. The aqueous detergent solution can comprise a variety of ingredients including anionic, nonionic or cationic surfactant materials, other ingredients, etc.

One anionic surfactant useful for detersive purposes can also be included in the compositions hereof. These can include salts (including, for example, sodium, potassium, ammonium, and substituted ammonium salts such as mono-, di- and triethanolamine salts) of soap, C₉-C₂₀ linear alkylbenzenesulfonates, C₈-C₂₂ primary or secondary alkanesulfonates, C₈-C₂₄ olefinsulfonates, sulfonated polycarboxylic acids prepared by sulfonation of the pyrolyzed product of alkaline earth metal citrates. C₈-C₂₄ alkylpolyglycolethersulfates (containing up to 10 moles of ethylene oxide); alkyl glycerol sulfonates, fatty acyl glycerol sulfonates, fatty oleyl glycerols sulfates, alkyl phenol ethylene oxide ether sulfates, paraffin sulfonates, alkyl phosphates, isethionates such as the acyl isethionates, acyl laurates, fatty acid amides of methyl tauride, alkyl succinamates and sulfosuccinates, monoesters of sulfosuccinates (especially saturated and unsaturated C₁₂-C₁₈ monoesters) and diesters of sulfosuccinates (especially saturated and unsaturated C₆-C₁₂ diesters), acyl sarcosinates; sulfates of alkylpolysaccharides such as the sulfates of alkylpolyglucosode (the nonionic nonsulfated compounds being described below), branched primary alkyl, sulfates, and fatty acids esterified with isethionic acid and neutralized with sodium hydroxide. Resin acids and hydrogenated resin acids are also suitable, such as rosin, hydrogenated rosin, and resin acids and hydrogenated resin acids present in or derived from tall oil.

Another type of anionic surfactant which can be utilized encompasses alkyl ester sulfonates. Alkyl ester sulfonate surfactants hereof include linear esters of C₈-C₂₀ carboxylic acids (i.e., fatty acids) which are sulfonated with gaseous SO₃ according to “The Journal of the American Oil Chemists Society” 52 (1975), pp. 323-329. Suitable starting materials would include natural fatty substances as derived from tallow, palm oil, etc. Alkyl sulfate surfactants hereof are water soluble salts or acids of the formula ROSO₃M wherein R preferably is a C₁₀-C₂₄ hydrocarbyl, preferably an alkyl or hydroxyalkyl having a C₁₀-C-₂₀ alkyl component, more preferably a C₁₂-C₁₈ alkyl or hydroxyalkyl, and M is H or a cation, e.g., an alkali metal cation (e.g., sodium, potassium, lithium), or ammonium or substituted ammonium (e.g., methyl-, dimethyl-, and trimethyl ammonium cations and quaternary ammonium cations such as tetramethylammonium and dimethyl piperdinium cations and quaternary ammonium cations derived from alkylamines such as ethylamine, diethylamine, triethylamine, and mixtures thereof, and the like). Alkyl alkoxylated sulfate surfactants hereof are water soluble salts or acids of the formula RO(A)ₘSO₃—M⁺ wherein R is an unsubstituted C₁₀-C₂₄ alkyl or hydroxy alkyl group having a C₁₀-C₂₄ alkyl component, preferably C₁₂-C₂₀ alkyl or hydroxyalkyl, more preferably C₁₂-C₁₈ alkyl or hydroxyalkyl, A is an ethoxy or propoxy unit, m is greater than zero, typically between about 0.5 and about 6, more preferably between about 0.5 and about 3, and M is H or a cation which can be, for example, a metal cation (e.g., sodium, potassium, lithium, calcium, magnesium, etc.). ammonium or substituted-ammonium cation. Alkyl ethoxylated sulfates as well as alkyl propoxylated sulfates are contemplated herein. Specific examples of substituted ammonium cations include methyl-, dimethyl-, trimethyl-ammonium cations and quaternary ammonium cations such as tetramethyl-ammonium and dimethyl piperdinium cations and those derived from alkylamines such as ethylamine, diethylamine, triethyl-amine, mixtures thereof, and the like.

Conventional, nonionic detersive surfactants for purposes of this invention include the polyethylene, polypropylene, and polybutylene oxide condensates of alkyl phenols. In general, the polyethylene oxide condensates are preferred. These compounds include the condensation products of alkyl phenols having an alkyl group containing from about 6 to about 12 carbon atoms in either a straight chain or branched chain configuration with the alkylene oxide. In a preferred embodiment, the ethylene oxide is present in an amount equal to from about 5 to about 25 moles of ethylene oxide per mole of alkyl phenol. Commercially available nonionic surfactants of this type include Igepal™ CO-630, marketed by the GAF Corporation; and Triton™ X-45, X-114, X-100, and X-102, all marketed by the Rohm & Haas Company. Nonionic surfactants also include the condensation products of aliphatic alcohols with from about 1 to about 25 moles of ethylene oxide. The alkyl chain of the aliphatic alcohol can either be straight or branched, primary or secondary, and generally contains from about 8 to about 22 carbon atoms. Particularly preferred are the condensation products of alcohols having an alkyl group containing from about 10 to about 20 carbon atoms with from about 2 to about 10 moles of ethylene oxide per mole of alcohol. Examples of commercially available nonionic surfactants of this type include Tergitol™ 15·5·9 (the condensation product of C₁₁-C₁₅ linear alcohol with 9 moles ethylene oxide), Tergitol™ 24-L-6 NMW (the condensation product of C₁₂-C₁₄ primary alcohol with 6 moles ethylene oxide with a narrow molecular weight distribution), both marketed by Union Carbide Corporation; Neodol™ 45-9 (the condensation product of C₁₄-C₁₅ linear alcohol with 9 moles of ethylene oxide), Neodol™ 23-6.5 (the condensation product of C₁₂-C₁₃ linear alcohol with 6.5 moles of ethylene oxide), Neodol™ 45.7 (the condensation product of C₁₄-C₁₅ linear alcohol with 7 moles of ethylene oxide), Neodol™ 45.4 (the condensation product of C₁₄-C₁₅ linear alcohol with 4 moles of ethylene oxide), marketed by Shell Chemical Company, and Kyro™ EOB (the condensation product of C₁₃-C₁₅ alcohol with 9 moles ethylene oxide), marketed by The Procter & Gamble Company. The condensation products of ethylene oxide with a hydrophobic base formed by the condensation of propylene oxide with propylene glycol can also be used. The hydrophobic portion of these compounds preferably has a molecular weight of from about 1500 to about 1800 and exhibits water insolubility. The addition of polyoxyethylene moieties to this hydrophobic portion tends to increase the water solubility of the molecule as a whole, and the liquid character of the product is retained up to the point where the polyoxyethylene content is about 50% of the total weight of the condensation product, which corresponds to condensation with up to about 40 moles of ethylene oxide. Examples of compounds of this type include certain of the commercially available Pluronic™ surfactants, marketed by BASF.

Cationic detersive surfactants can also be included in detergent compositions of the present invention. Cationic surfactants include the ammonium surfactants such as alkyldimethylammonium halogenides, and those surfactants having the formula:

[R²(OR³)_{y}][R⁴(OR³)ₓ]₃R³N⁺X⁻;

wherein R² is an alkyl or alkyl benzyl group having from about 8 to about 18 carbon atoms in the alkyl chain, each R³ is selected from the group consisting of:

—CH₃CH₂—, —CH₂CH(CH₃)—, —CHCH(CH₂OH)—, —CH₂CH₂CH₂—

and mixtures thereof; each R⁴ is selected from the group consisting of C₁-C₄ alkyl, C₁-C₄ hydroxylalkyl, benzyl ring structures formed by joining the two R⁴ groups, —CH₂CHOH——CHOHCOR⁶CHOHCH₂OH wherein R⁶ is any hexose or hexose polymer having a molecular weight less than about 1000, and hydrogen when y is not 0; R⁵ is the same as R⁴ or is an alkyl chain wherein the total number of carbon atoms of R² plus R⁵ is not more than about 18; each y is from 0 to about 10 and the sum of the y values is from 0 to about 15; and X is any compatible anion.

The detergent compositions of the present invention comprises a liquid carrier, e.g., water, preferably a mixture of water and a C₁-C₄ monohydric alcohol (e.g., ethanol, propanol, isopropanol, butanol, and mixtures thereof), with ethanol being the preferred alcohol.

A wide variety of other ingredients useful in detergent compositions can be included in the compositions hereof, including other active ingredients, carriers, processing aids, dyes or pigments, perfumes, solvents for liquid formulations, hydrotropes (as described below), etc.

Liquid detergent compositions can contain water and other solvents. Low molecular weight primary or secondary alcohols exemplified by methanol, ethanol, propanol, and isopropanol are suitable. Monohydric alcohols are preferred for solubilizing surfactant, but polyols such as those containing from about 2 to about 6 carbon atoms and from about 2 to about 6 hydroxy groups (e.g., propylene glycol, ethylene glycol, glycerin, and 1,2-propanediol) can also be used.

The detergent compositions hereof will preferably be formulated such that during use in aqueous cleaning operations the wash water will have a pH of between about 6.5 and about 11, preferably between about 7.5 and about 10.5. Liquid product formulations preferably have a (10% dilution) pH between about 7.5 and about 10.0, more preferably between about 7.5 and about 9.0 Techniques for controlling pH at recommended usage levels include the use of buffers, alkali, acids, etc., and are well known to those skilled in the art.

Solid Unit

A chlorine containing bath having an indicator dye can be made by introducing into water, a solid unit such as a tablet, a pellet or other small compressed solid cast unit or extruded material. The unit containing a chlorine is formulated to contain solid active chlorine material and the indicator dye. The solid unit can be configured with sufficient material to treat an appropriate amount of water to form the indicated chlorine containing aqueous solution. The size of the tablet, pellet or solid unit can range from greater than 200 milligrams to include sizes that can be as much as 100 grams depending on the amount of water. Typically, the materials are used such that a unit has about 1 to 50 grams preferably 1 to 20, typically 4 to 8 grams of material in a single unit and can be used to treat about 1 liter of water or more, a typical sink volume of 1 to 100 preferably 10 to 50 liters.

The preferred solid units of the invention typically contain a solid chlorine source and a dye. Typical solid chlorine sources include sodium dichloroisocyanurate dihydrate, chlorinated sodium phosphate, calcium hypochlorite, chloramines and other well known and available sources of chlorine and solid particulate or granular form. Useful dyes include those set forth above in the application. The solid pellets of the invention can also contain solid organic or inorganic components that can control pH of the chlorine solution.

In the solid unit aspect of the invention, the physical form of the dye is important to the stability of the dye in contact with the chlorine source. Most dyes comprise complex organic molecules that are easily oxidized by compounds such as active chlorine sources. We have found that a dye composition, in the form of a particle or granule, having a particle size greater than about 200 microns, preferably greater than about 500 microns, most preferably greater than about 700 microns can be used in the solid unit and remain stable for indefinite periods. We believe the particle size of a granular dye reduces the tendency of the dye to react with the active chlorine material in the solid unit. This is particularly true in the dry systems made in this invention.

The solid units of the invention are typically made with little or no free water or water added. Free water within the solid unit can provide a medium for reaction between the chlorine source and the dye material, even if formulated or formed from a granular dye. Accordingly, the solid units of the invention have little or no free water present. Water can be present in the solid unit in the form of water of hydration as long as such water is not released from the hydration location into the solid unit for the purpose of providing a medium for reaction. Water of hydration, for example, of the sodium dichloroisocyanurate dihydrate remains securely bonded to the chlorinated molecule and does not typically act to reduce compatibility. Other hydrated materials can be used in the solid unit of the invention. For example, extender salt hydrates may be present in the solid unit for the purpose of diluting the chlorine source, modifying dissolution rates, changing the size of the solid unit for the purpose of acting as a binder for the solid unit or further purposes.

In the typical solid units of the invention, the weight ratio between the chlorine source and the dye will be typically about 1 to about 200 grams of chlorine source per gram of dye.

The solid units of the invention can be made using a variety of solids forming technology. The only limitation on such technology is the need to avoid forming substantial quantities of free water remaining in the solid unit. Accordingly, preferred modes for forming the solid unit of the invention include casting the solid units from a castable, typically non-aqueous liquid, or by forming pellets or tablets by compressing powder mixture in tablet or pelletizing equipment under sufficient pressure and in the pressure of optional binders to form a useful solid unit. In forming the solid units of the invention, a mold, a tablet or pellet press equipment can be used to form a tablet having dimensions of about 2 to 50 millimeters in diameter, preferably 5 to 25 millimeters in diameter. Tablet thicknesses can range from about 2 to 20 millimeters. Most preferred diameters range from about 10 to 25 millimeters.

A useful 20 millimeter tablet can be made using a tablet press that can exert 2 tons force to particulates in a tableting dye. In such a process, a quantity of a mixture of the solid chlorine source and granular dye can be placed manually or in an automated mode into the tablet dye and compressed for dwell time of 1 to 30 seconds to a pressure of ½ to 15 tons per square inch. The tableting dyes can be entirely cylindrical or can have a concave or opaque top or bottom surface to obtain a desired tablet shape. Sufficient pressure is placed on the particulate to achieve a hardness of greater than about 50 psi, typically 60 to 100 psi.

The tablets of the invention can be made using conventional tableting technology. In manufacturing the tablets of the invention, dry, granular or powder material are combined in typical powder blending equipment to ensure any uniform mixture of ingredients that typically include the granular dichlorodiisocyanurate chlorine source, the dye in a granular form and often a processing aid or dye release material. Any conventional tableter can be used that can form a table of the appropriate dimensions. The preferred tablet dimensions is about 1.5 to 2.5 centimeters in diameter with a thickness of about 1 to 2 centimeters. Typical processing conditions involve a tableting pressure of at least 5 tons or more, tablet formation occurring in 1 to 5 seconds, typically 2 to 3 seconds.

The compositions and tablets of the invention can be used in a variety of ways. The material can simply be added directly to a sink when the color is depleted. Further, the materials can be added from a dispenser that can dispense either a measured portion of the powdered material or a single tablet of the tableted materials. The tablets can be shaped to fit in a tablet dispenser with a lock-out feature. The shape of the tablets can be such that only the tablet shapes can fit the dispenser profile. In such a way, only the appropriate tablets can be placed into the dispenser to avoid either waste of material or hazardous combinations of ingredients.

Generic Formulae Chlorine Solid Unit or Powdered Concentrate

| | Useful | Preferred |
|---|---|---|
| | Weight | Weight |
| Ingredient | Percentage | Percentage |
| Chlorinated encapsulate ACP | 5 to 99.9 | 50 to 96 |
| FD & C red dye No. 40 | 0.001 to 5 | 0.5 to 0.09 |
| Source of acid | 0 to 10 | 0.01 to 5 |
| tableting aid | 0 to 0.1 | 0 to 0.01 |

The above discussion of the components of the invention provides a basis for understanding the compositions of the invention and the useful process steps. The following example and data illustrate the utility of the invention and contain a best mode.

### EXAMPLE I

Powdered Acidic Formulations

| | #1 | #2 | #3 | #4 | #5 | #6 |
|---|---|---|---|---|---|---|
| Component | (wt- %) | (wt- %) | (wt- %) | (wt- %) | (wt- %) | (wt- %) |
| ACP¹ | 33.60 | 34.90 | 9.50 | 0 | 67.20 | 27.57 |
| SAPP² | 14.10 | 56.40 | 25.70 | 14.1 | 28.24 | 0 |
| anhydrous | 2.11 | 8.40 | 0 | 26.6 | 4.20 | 5.97 |
| citric acid | | | | | | |
| FD&C Dye³ | 0.14 | 0.20 | 0.04 | 0.07 | 0.28 | 0.20 |
| propylene | 0.17 | 0.10 | 1.00 | 0.11 | 0.08 | 0.10 |
| glycol | | | | | | |
| sodium sulfate | 49.88 | 0 | 46.46 | 9.12 | 0 | 0 |
| MSP⁴ | 0 | 0 | 17.30 | 0 | 0 | 66.16 |
| chlorinated | 0 | 0 | 0 | 50.0 | 0 | 0 |
| TSP⁵ | | | | | | |

¹Encapsulated sodium dichloro-s-triazinetrione dihydrate.
²Sodium acid pyrophosphate.
³FD&C red #40, FD&C blue #1, etc.
⁴Monosodium phosphate.
⁵Chlorinated trisodium phosphate.

Formulations 1 and 3 listed above were made and placed into a 120° F. (49° C.) oven for long term stability testing. The formulations were monitored weekly for available chlorine levels and for color stability. Duplicates were made of formulations and 3 which differed only in using non-encapsulated sodium dichloro-s-triazinetrione as the chlorine source. After 8 weeks, all of the formulations having encapsulated chlorine sources retained acceptable active levels. The two formulations lacking an encapsulated chlorine source lost their efficacy after only 1 week. The active chlorine source bleached the dye.

### EXAMPLE II

Powdered Chlorine Concentrate

| Ingredient | Percentage |
|---|---|
| Chlorinated encapsulate ACP | 33.6 |
| FD & C red dye No. 40 | 0.14 |
| Citric acid | 2.1 |
| Sodium acid pyrophosphate | 14.1 |
| (SAPP) | |
| Sodium sulfate | 49.9 |

Using Example II, a sanitizing solution containing 30 ppm chlorine and 10 ppm dye at pH about 7 provided active sanitizing with solution color lasting about two hours. At a lower pH, between 5 and 6, a sanitizing solution containing 30 ppm chlorine and 10 ppm dye lasted approximately four hours. In both cases, substantial sanitizing activity was observed without corrosion or chlorine gassing.

### EXAMPLE III

A dye and chlorine stability test was performed using an initial solution containing 100 ppm chlorine and 1 ppm of FD&C Red #40 dye. CDB (Sodium dichloroisocyanurate dihydrate) was used as the chlorine source and the tests were conducted with an initial temperature of 80° F. (26.7° C.). The following data demonstrate the effects of pH on dye and chlorine stability:

Results

| pH buffered | Time | | Available Chlorine |
|---|---|---|---|
| at | (hours) | Color/Appearance | (ppm) |
| 2 | 0.0 | color gone immediately | 10-50 |
| 4 | 2.5 | color gone | 80-100 |
| 6 | 5.0 | slightly visible | 100 |
| 8 | 0.25 | color gone | 100 |
| 10 | 0.0 | color gone immediately | 100 |
| 12 | 0.0 | color gone immediately | 100 |
| 2 | 175 | no color | 0 |
| 4 | 175 | no color | 0 |
| 6 | 175 | no color | 80-100 |
| 8 | 175 | no color | 50-100 |
| 10 | 175 | no color | 10 |
| 12 | 175 | no color | 10 |

Additional formulations were tested at an active chlorine level of 100 ppm and at pH levels which were buffered to between 2 and 12. Each formulation included 1 ppm FD&C Red #40 dye and began at 80° F. (27° C.).

| | | Time needed for |
|---|---|---|
| pH buffered | Initial | solution to become |
| at | Color/Appearance | colorless (hours) |
| 2 | colorless | none |
| 4 | visible color | 2.5 |
| 6 | visible color | 4.0 |
| 8 | visible color | 0.25 |
| 10 | colorless | none |
| 12 | colorless | none |
| 5.8 | visible color | 5.0 |
| 5.4 | visible color | 15 |

Several conclusions can be drawn from the data above: With a pH range of 5.8 to 6.3, the color lasts 4.5 to 5.0 hours in the sink. With a pH range of 5.3 to 5.6 the color lasts 14 to 16 hours in a bulk container which can be used through spray bottle in a daily sanitizing regimen. The sanitizer materials are to be replaced daily. A chlorine encapsulate like ACP or Enforcer RC is required for dye stability in the powder. In the first table, which indicates time needed for the color to disappear, several additional comments can be made. At low pH, pH 2 to pH 4, the dye is destroyed because of the pH. Additionally, the solution is a skin irritant. Conversely, at high pH, that is pH 8 and greater, the dye is destroyed by the OCl⁻¹ ion.

### EXAMPLE IV

A test was conducted with several solutions at active levels ranging from 10 ppm to 100 ppm active chlorine. Each solution started with 1 ppm FD&C red #40 and was buffered to a pH of 5.8.

| ppm available | | Time needed for solution to |
|---|---|---|
| chlorine | Initial Color/appearance | become colorless (hours) |
| 10 | visible | more than 6.0 |
| 30 | visible | 6.0 |
| 50 | visible | 5.0 |
| 80 | visible | 4.5 |
| 100 | visible | 3.0 |

A test was also conducted with 4 solutions by varying the level of dye (FD&C red #40) from 0.1 to 0.4 wt-%. Each solution was buffered to a pH of 5.8 and had an initial active level of 100 ppm available chlorine. As expected, there is a linear relationship between dye concentration and color longevity.

As a result, a sanitizing solution can be made visible based on the composition of the concentrate. The length of time that the visibility or color of the solution lasts can be controlled by varying the percentages of the dye, the level of active and the pH/buffer component. The pH/buffer component has the greatest effect, while the dye and active can be used for fine-tuning.

### EXAMPLE V

Example V involves a liquid co-system. This is a two-part system. The first solution contains sufficient NaOCl into the sink to produce 100 ppm available chlorine and sufficient H₃PO₄ to produce a pH between 5 and 6, 1.0% of FD&C RED Dye #40 and color lasting between 2 and 6 hours. The second solution contains sufficient NaOCl in the sink to produce 100 ppm available chlorine, 20.0% of a 75% active aqueous H₃PO₄, 1.0% of FD&C RED Dye #40 and 79.0% of water. The color lasts for at least one hour.

These formulations show that non-encapsulated liquid sources of chlorine can be used with useful results.

### EXAMPLES VI-IX

A variety of formulations have been found to be useful in both these methods in which the formulations are diluted with water and used. These formulations are disclosed in the tables below.

Sink Formula with a 3 to 6 hour life

| Formula | Raw Material | % | ppm(use) | pak-oz. |
|---|---|---|---|---|
| Ex VI | Encapsulated chlorine ACP | 33.600 | 100 | 1.0 |
| | SAPP¹ | 14.100 | | |
| | Citric Acid (anh) | 2.110 | | |
| | Panodan² | 0.166 | | |
| | FD & C RED #40 | 0.140 | | |
| | NaCl (diluent also Na₂SO₄) | 49.884 | | |
| Ex VII | Encapsulated chlorine ACP | 67.20 | 100 | 0.5 |
| | SAPP | 28.20 | | |
| | Citric Acid (anh) | 4.22 | | |
| | Panodan | 0.10 | | |
| | FD & C RED #40 Gran | 0.28 | | |
| Ex VIII | Encapsulated chlorine ACP | 10.100 | | |
| | SAPP | 14.100 | | |
| | Citric Acid (anh) | 2.110 | | |
| | Panodan | 0.166 | | |
| | FD & C RED #40 | 0.140 | | |
| | NaCl flake | 73.384 | | |
| Ex IX | Encapsulated chlorine ACP | 20.200 | | |
| | SAPP | 28.200 | | |
| | Citric Acid (anh) | 4.220 | | |
| | Panodan | 0.166 | | |
| | FD & C RED #40 | 0.280 | | |
| | NaCl flake | 46.934 | | |

¹Sodium acid pyrophosphate.
²Diacetyl tartaric acid ester of long chain C₁₆₋₁₈ fatty acid mixed monoglycerides and diglycerides.

Spray Bottle Formula with 3 to 18 hour life

| Formula | Raw Material | % | ppm(use) | pak-oz. |
|---|---|---|---|---|
| Ex X | Enforcer RC³ (encapsulated Cl₂) | 11.20 | 100 | 1.0 |
| | SAPP | 14.10 | | |
| | Citric Acid (anh) | 00.80 | | |
| | FD & C RED #40 | 00.40 | | |
| | NaCl flake | 73.36 | | |
| | Panodan | 00.50 | | |
| Ex XI | Enforcer RC | 22.40 | 100 | 0.5 |
| | SAPP | 28.20 | | |
| | Citric Acid (anh) | 01.60 | | |
| | FD & C RED #40 | 00.08 | | |
| | Panodan | 00.75 | | |
| | Fill (powder diluent) | 46.97 | | |
| Ex XII | Enforcer RC | 03.40 | 30 | 1.0 |
| | SAPP | 14.10 | | |
| | Citric Acid (anh) | 00.08 | | |
| | FD & C RED #40 | 00.04 | | |
| | Panodan | 00.50 | | |
| | Fill (powder diluent) | 81.88 | | |

Surprisingly, we have found, under the conditions of use shown in the Examples above, that a dye, typically considered to be unstable in the presence of strong oxidants such as halogen bleaches, can remain stable for a sufficient period of time to be used as an indicator of an oxidative quality of the solution and/or the efficacy of a sanitizer solution. The use of an encapsulated chlorine source in a powdered concentrate appears to be important in maintaining and extending the stability. The stability permits the use of such a dye with such an oxidative halogen bleach in a hard surface sanitizing method and a hand warewashing method. In hand ware washing, the ware is first washed with a typical surfactant system and then sanitized in the dye containing halogen solution. We have found that the indicator can be used to show the effective concentration of the chlorine source and can suggest the appropriate time for replacing the chlorine bleach solution at intervals which results in the efficient use of the sanitizer solution. If the solutions were replaced too early, the chlorine bleach materials can be wasted. If the solutions were replaced after too long an interval, solutions would be depleted of active chlorine species and would not bleach or sanitize the ware. The overall process of the invention produces clean bleached and sanitized ware in a handwashing system without wasted chlorine bleach materials.

### EXAMPLE XIII

Experimental work was conducted in order to demonstrate antimicrobial or sanitizing activity of the materials having the dye indicator content. Testing was conducted in accordance with the official methods of analysis for the “available chlorine germicidal equivalent concentration” test, AOAC, Fifteenth Edition, 1990, Chapter 6, Section 955.16, pp. 137-138, per TEC-TM-001. Following the provisions of that test, five sanitizer solutions were formulated having a chlorine concentration that ranged from about 9.8 to about 110 ppm active chlorine. The solutions were made from concentration mixed at about 0.75 gram per liter of water or about one ounce per ten gallons. The sanitizers were formulated with a pH between 6 and 7. The solutions were prepared for the purpose of determining chlorine longevity and sanitizing efficacy. The following table shows the formula and the chlorine concentration. The test organism used was Staphylococcus auras, ATCC No. 6538.

Formulae and Cl₂ concentration

| FORMULATION | Cl₂ Conc. (ppm) |
|---|---|
| A | 100 |
| B | 78 |
| C | 48 |
| D | 31 |
| E | 9.8 |

Following the protocol set forth above, the following results were obtained:

Microbiological test results

| | ppm | 1 | 2 | 3 | 4 | 5 | 6 | 7 | 8 | 9 | 10 |
|---|---|---|---|---|---|---|---|---|---|---|---|
| Chlorine Std. | 52 | − | − | + | + | + | + | + | + | + | + |
| A | 110 | − | − | − | + | + | + | + | + | + | + |
| B | 78 | − | − | − | + | + | + | + | + | + | + |
| C | 48 | − | − | + | + | + | + | + | + | + | + |
| D | 31 | − | + | + | + | + | + | + | + | + | + |
| E | 9.8 | + | + | + | + | + | + | + | + | + | + |

Results: (+ means positive growth, − means negative growth)

Results were recorded after approximately 48 hours incubation at 37° C.

The bacterial efficacy of a sample must be equivalent to, or greater than, the 50 ppm chlorine standard to be certified by the USDA. Equivalency is met when the sample tubes have an absence in growth in as many tubes as the chlorine standard. The five experimental sanitizers exhibited bactericidal efficacy approximating the expected results from chlorine standards prepared at those concentrations. Thus, while passing the Available Chlorine test against S. aureus, the sanitizer formulations showed no enhancement of antibacterial properties over those of our current formula as expected with this test.

### EXAMPLE XIV

A similar set of chlorine base sanitizer solutions were made using compositions made from chlorinated isocyanurate or chlorinated trisodium phosphate. The chlorine concentration ranged from 10 to 30 ppm. These solutions were tested for sanitizing capacity and chlorine stability. The following test shows the results:

ACID SANITIZER MICROBIOLOGY TEST

| | | | | Chlor- | Chlor- | Chlor- |
|---|---|---|---|---|---|---|
| Chlorine | | | | ine | ine | ine |
| Based | | | | (ppm) | (ppm) | (ppm) |
| Sanitizer | | | | Conc. | Conc. | Conc. |
| Chlorine | Source of | % | | 0 | 4 | 24 |
| ppm | Chlorine | Reduction | pH | Time | Hours | Hours |
| 10 | CDB | 100.0000 | 4.55 | 9.9 | 8.5 | 5.67 |
| | Chlorinated | | | | | |
| | Isocyanurate | | | | | |
| 10 | CDB | 99.9999 | 4.55 | 9.9 | 8.5 | 5.67 |
| | Chlorinated | | | | | |
| | Isocyanurate | | | | | |
| 30 | CDB | 100.0000 | 4.78 | 26.9 | 22.69 | 19.85 |
| | Chlorinated | | | | | |
| | Isocyanurate | | | | | |
| 30 | CDB | 100.0000 | 4.78 | 26.9 | 22.69 | 19.85 |
| | Chlorinated | | | | | |
| | Isocyanurate | | | | | |
| 12 | Chlorinated | 100.0000 | 5.13 | 12.7 | 9.93 | 5.67 |
| | TSP | | | | | |
| 12 | Chlorinated | 100.0000 | 5.13 | 12.7 | 9.93 | 5.67 |
| | TSP | | | | | |
| Inoculum | 2.2E+9 | | | | | |
| Control | 21.8E+6 | | | | | |

This table demonstrates that the chlorine concentration can last more than 24 hours and provide adequate microbial control.

### EXAMPLE XV

The formulations listed below were submitted for microbiological efficacy testing according to the AOAC Germical and Detergent Sanitizers Method.

| | #13 | #14 | #15 | #16 | #17 |
|---|---|---|---|---|---|
| Component | (wt- %) | (wt- %) | (wt- %) | (wt- %) | (wt- %) |
| sodium sulfate | 49.90 | 56.58 | 66.68 | 50.00 | 0 |
| SAPP¹ | 14.10 | 14.10 | 14.10 | 28.20 | 56.40 |
| anhydrous citric | 2.11 | 2.11 | 2.11 | 2.00 | 8.40 |
| acid | | | | | |
| propylene glycol | 0.15 | 0.17 | 0.17 | 0.56 | 0.10 |
| FD&C red #40 | 0.14 | 0.14 | 0.14 | 0.10 | 0.20 |
| ACP² | 33.60 | 26.90 | 16.80 | 19.14 | 34.90 |

¹Encapsulated sodium dichloro-s-triazinetrione dehydrate.
²Sodium acid pyrophosphate.

The following results were obtained using both S. aureus (ATCC 6538) and E. coli (ATCC 11229).

| Formulation | | Average Survivors | Percent |
|---|---|---|---|
| # | Test Culture | (cfu/ml) | Reduction |
| 13 | S. aureus | 20 | 99.999 |
| 14 | S. aureus | 5 | 99.999 |
| 15 | S. aureus | <10 | >99.999 |
| 16 | S. aureus | <10 | >99.999 |
| 17 | S. aureus | <10 | >99.999 |
| 13 | E. coli | <10 | >99.999 |
| 14 | E. coli | <10 | >99.999 |
| 15 | E. coli | <10 | >99.999 |
| 16 | E. coli | <10 | >99.999 |
| 17 | E. coli | <10 | >99.999 |

### EXAMPLE XVII

In an appropriate mixing container, 113.3 grams of sodium dichloro-isocyanurate dihydrate is combined with about 1 gram of a FD&C Red # 40 granular dye having a powder size of about 700 microns. The blended powder was introduced into an automated tablet press forming a tablet ¾ inch (19 mm) in diameter. About 6.89 grams of the blended powdered material was introduced into the dye and compressed into the tablet using about 2 tons pressure. The tablet formed quickly and was hard and not fryable. The hardness was measured within a range of about 60 to about 90 psi.

The tableted product produced in the Example was used in forming an active chlorine containing aqueous solution in a sink. The solution is used over a period of 4 hours. The solution is discarded after the dye disappears indicating that the typical lifetime of the solution has ended.

### EXAMPLES XVIIIA and XVIIIB

### Tablet Examples

Using the procedure of Example XVII, a 10 gram tablet was made using the following formulas.

| Ingredients | A (wt %) | B (wt %) |
|---|---|---|
| Granular sodium | 99.56 | 97.57 |
| dichloroisocyanurate | | |
| dihydrate | | |
| FD & C #40 (Granular) | 0.44 | 1.43 |
| Sodium Stearate | 0.0 | 1.0 |

### EXAMPLES XIXA and XIXB

### Tablet Examples

Using the procedure of Example XVII, a 6.8 gram tablet was made using the following formulas.

| Ingredients | A (wt %) | B (wt %) |
|---|---|---|
| Granular sodium | 99.56 | 97.57 |
| dichloroisocyanurate | | |
| dihydrate | | |
| FD&C #40 (Granular) | 0.44 | 1.43 |
| Sodium Stearate | 0.0 | 1.0 |

The tableted products of Example XVIII and XIX were used in a sanitizing solution at a ratio of one tablet in a 10 gallon volume of water. The pH was about 6.0 and produced at least 100 ppm of active chlorine in the water until the dye color was depleted. The tablet was also tested for stability. At ambient temperature, the materials lost no chlorine or dye activity over a six month period of storage at typical ambient conditions at ambient temperature of about 70-75° F. with ambient relative humidity. In a five month extreme environment test, the tablets had no substantial loss of chlorine or dye activity over five months held at a temperature between 112°-127° F.

The above specification provides the basis for understanding compositions that can be used in formulating the materials used in the process of the invention. The example and data also provide a basis to understand a specific embodiment of the invention and disclose the best mode. Since many embodiments can be made without departure from the spirit and scope of the invention, the invention is found in the claims hereinafter appended.

We claim:

1. An active chlorine containing solid unit containing a chlorine source and an stable source of dye, the solid unit comprising:
(a) about 10 to about 200 parts by weight of a solid active source of chlorine per each part of dye; and
(b) a source of a dye, the dye comprising a particulate dye having a minimum particle size of about 200 microns, wherein the solid unit comprises a major dimension greater than about 2 millimeters and a weight greater than about 2 grams, the solid unit substantially free of an amount of free water sufficient to act as a reaction medium between the solid chlorine source and the dye.

2. The solid unit of claim 1 wherein the dye comprises a dye with a minimum particle size of about 500 microns and a density less than 0.9 gram-cm⁻³.

3. The solid unit of claim 1 wherein the solid unit comprises a cylindrical tablet having a diameter of about 4 to 75 millimeters and a thickness of about 1 to 25 millimeters.

4. The solid unit of claim 1 wherein the solid chlorine source comprises an alkali metal dichloroisocyanurate dihydrate.

5. The solid unit of claim 1 wherein the solid unit comprises a spheroid having a major dimension of about 5 to 60 millimeters and one perpendicular dimension of about 1 to 50 millimeters.

6. The solid unit of claim 4 wherein the solid chlorine source comprises an encapsulated alkali metal dichloroisocyanurate dihydrate.

7. The solid unit of claim 1 wherein the dye comprises a granular dye having a particle size greater than about 600 microns and a density less than about 0.85 grams-cm⁻.

8. A method of using the solid unit of claim 1 in a cleaning or sanitizing operation, the method comprises:
(a) placing a solid unit, comprising an active chlorine source and a dye, in a volume of an aqueous liquid in a container, the weight ratio of the solid to the aqueous solution being about 0.1 to 20 grams per liter of water to form a dye colored, active-chlorine solution;
(b) contacting ware with the aqueous active-chlorine solution during cleaning or sanitizing operations for a period of up to 4 hours and after detecting a color change, either replacing the aqueous solution or replenishing the aqueous solution with additional chlorine source.

9. A particulate composition forming an aqueous solution having an active chlorine source and a dye, the powdered concentrate comprising:
(a) about 1 to 90 wt % of an encapsulated source of halogen; and
(b) an effective amount of dye;
wherein the concentrate is has substantially no free water, has an extended shelf life of greater than one month and when added to an aqueous diluent provides a dye that indicates the presence of an active halogen concentration for a predetermined time.

10. The composition of claim 9 wherein the source of halogen comprises a source of chlorine.

11. The composition of claim 10 wherein the source of chlorine comprises chloroisocyanurate compound.

12. The composition of claim 9 which also comprises an acid source to obtain a pH less than 7 in the aqueous solution.

13. The composition of claim 9 wherein the indicator comprises FD&C dye No. 40.

14. The composition of claim 9 wherein the indicator comprises FD&C dye No. 3.

15. The composition of claim 12 wherein the acid source comprises a solid acid.

16. The composition of claim 9 wherein the acid salt comprises sodium dihydrogen phosphate, sodium hydrogen tartrate, sodium hydrogen sulfate, or mixtures thereof.

17. The composition of claim 9 wherein the builder salt comprises sodium sulfate, sodium carbonate, trisodium phosphate, sodium bicarbonate or mixtures thereof.

18. The composition of claim 9 wherein the concentration of dye in the concentrate is adjusted such that the dye color changes or is depleted during a useful predetermined period of time during which the sanitizer solution can be used for its intended purpose and maintain at least 50 ppm active chlorine.

19. An aqueous liquid cleaning or sanitizing composition containing a dye that indicates chlorine concentration, the liquid comprising a major proportion of an aqueous diluent, and
(a) a source of acid;
(b) an effective amount of a dye to obtain a colored solution for a predetermined period of time;
(c) an effective cleaning or sanitizing amount of a halogen bleach;
wherein the aqueous solution has a pH less than 7 and the dye color is depleted or changed before the concentration of halogen is depleted to less than 50 ppm from the solution.

20. The concentrate of claim 19 wherein the source of halogen comprises a source of chlorine.

21. The composition of claim 20 wherein the source of chlorine comprises a chloroisocyanurate compound.

22. The composition of claim 19 which also comprises a builder salt.

23. The composition of claim 19 wherein the indicator comprises FD&C dye No. 40.

24. The composition of claim 21 wherein the chlorine source comprises an encapsulated alkali metal dichloroisocyanurate dihydrate.

25. The composition of claim 19 wherein the acid source comprises a solid acid.

26. The composition of claim 19 wherein the acid salt comprises sodium dihydrogen phosphate, sodium hydrogen tartrate, sodium hydrogen sulfate, or mixtures thereof.

27. The composition of claim 19 wherein the builder salt comprises sodium sulfate, sodium carbonate, trisodium phosphate, sodium bicarbonate or mixtures thereof.

28. The composition of claim 19 wherein the concentration of dye in the concentrate is adjusted such that the dye color changes or is depleted during a useful period of time during which the sanitizer solution can be used for its intended purpose.

29. A method of cleaning or sanitizing hard surfaces comprising:
(a) contacting the hard surface with an aqueous solution comprising the composition of claim 9, forming a surface having the aqueous liquid comprising a halogen source; and
(b) removing the aqueous liquid halogen source.

30. A method of hand washing ware in a sink having two or more basins, using a stable dye in an aqueous oxidative chlorine based cleaner or sanitizer composition, the method comprising:
(a) contacting ware with an aqueous detergent in a first basin to remove soil, producing cleaned ware; and
(b) contacting the cleaned ware in a subsequent basin with an aqueous sanitizer solution comprising an effective amount of a chlorine source, the sanitizer solution additionally comprising a dye that is sufficiently stable in the aqueous solution to maintain at least some detectable color in the sanitizing solution after greater than 90% of the oxidizing species have been consumed.

31. The method of claim 30 wherein the chlorine source comprises an alkali metal hypochlorite.

32. The method of claim 31 wherein the hypochlorite sanitizer comprises sodium hypochlorite.

33. The method of claim 30 wherein the chlorine source comprises a chlorinated isocyanurate compound which generates hypochlorous acid at the pH.

34. The method of claim 30 wherein the cleaned ware is contacted with a potable water rinse to form a rinsed cleaned ware prior to contacting the rinsed cleaned ware with the sanitizing solution.

35. The method of claim 30 wherein the aqueous sanitizer solution has a pH of less than about 7, the pH selected such that the concentration of OCl⁻¹ is minimized and the concentration of HOCl is maximized.

36. The method of claim 34 wherein the cleaned ware is contacted with the aqueous rinse for approximately 1 to about 30 seconds and the rinsed cleaned ware is contacted with the aqueous sanitizing solution for about 1 to 30 seconds.

37. The method of claim 30 wherein the ware is contacted with mechanical action in the first basin with the aqueous detergent for sufficient amount of time to substantially remove food soil and the cleaned ware is contacted with the aqueous sanitizer solution for about 1 to about 30 seconds.

38. The method of claim 30 wherein the concentration of the chlorine source is about 1 to 100 parts per million in the solution.

39. The method of claim 30 wherein the indicator comprises FD&C Dye #40.

40. The method of claim 30 wherein the indicator comprises FD&C Dye #3.

41. The method of claim 30 wherein after the sanitizing step, the ware is permitted to dry without contact with mechanical action or an aqueous solution.

42. The method of claim 30 wherein the sanitizing solution is made by diluting a powdered solid comprising:
(a) about 1 to 90 wt % of an encapsulated chlorine source;
(b) about 0.01 to 1.0 wt % of a dye;
(c) about 0.5 to 20 wt % of an acid source; and
(d) a major portion of a builder salt.

43. The method of claim 42 wherein the encapsulated chlorine source comprises an encapsulated chloroisocyanurate compound.

44. The method of claim 42 wherein the encapsulated chlorine source comprises a particle of the chlorine source, a first inorganic layer and a second organic layer.

45. The method of claim 42 wherein the dye comprises FD&C dye No. 40.

46. The method of claim 42 wherein the acid salt comprises potassium dihydrogen phosphate, sodium hydrogen tartrate or mixtures thereof.

47. The method of claim 42 wherein the builder salt comprises sodium sulfate.

48. The method of claim 42 wherein the pH of the aqueous sanitizing solution is adjusted to a pH less than 7 and to a pH at which greater than about 80% of the oxidative species is in the form of HOCl and less than about 20% of the oxidative species is in the form of OCl⁻¹.

49. The method of claim 42 wherein the dye color is maintained in the aqueous sanitizing solution for a period of time of about 3 to 6 hours.

50. A sanitizing solution useful in sanitizing a surface, the solution comprising:
(a) a major proportion of an aqueous medium having a pH less than 7;
(b) about 1 to 90 wt % of a source of an encapsulated active chlorine source resulting in at least 100 ppm active chlorine;
(c) an effective amount of a dye; and
(d) a solid diluent or extender salt.

51. The composition of claim 50 wherein the composition additionally comprises an acid salt selected from the group consisting of sodium acid phosphate, sodium acid tartrate or mixtures thereof.
